(19) United States
(12) Patent Application Publication
(10) Pub. No.: US 2010/0036262 A1
(43) Pub. Date: Feb. 11, 2010

(54) FLUORESCENCE ENDOSCOPE APPARATUS

(75) Inventors: Toshiaki Watanabe, Tokyo (JP); Ryo Karasawa, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, Tokyo (JP)

(21) Appl. No.: 12/531,999

(22) PCT Filed: Mar. 14, 2008
(86) PCT No.: PCT/JP2008/054771

§ 371 Date: Sep. 18, 2009

(30) Foreign Application Priority Data
Mar. 20, 2007 (JP) 2007-073393

(51) Int. Cl. A61B 1/07; A61B 6/00

(52) U.S. Cl. 600/478; 600/178

(57) ABSTRACT

The effect of noise light originating in a light guide portion is removed by simple calculations, and a clear fluorescence image that facilitates distinction between lesion tissue and normal tissue is acquired. Provided is a fluorescence endoscope apparatus including an insertion portion inserted into a body cavity; a light source unit that is disposed at a base end of the insertion portion and that emits excitation light and reference light that contains at least a part of the wavelength band of fluorescence produced by the excitation light; a light guide portion that guides the excitation light and the reference light emitted from the light source unit to a distal end of the insertion portion; an irradiation control unit that switches between a first irradiation state in which the excitation light guided by the light guide portion is radiated onto an inner wall of the body cavity and a second irradiation state in which the reference light is radiated onto the inner wall of the body cavity; an image-acquisition unit that acquires reflected light of the reference light and the fluorescence returning from the inner wall of the body cavity to the insertion portion; and an image computing unit that generates a fluorescence image signal by calculating the difference between a first image-acquisition signal acquired by the image-acquisition unit in the first irradiation state and a second image-acquisition signal acquired in the second irradiation state.

### TECHNICAL FIELD

The present invention relates to a fluorescence endoscope apparatus.

### BACKGROUND ART

In the related art, diagnostic techniques for finding affected areas by endoscopic observation utilizing fluorescence produced by biological tissue (autofluorescence) or fluorescence produced by fluorescent agents that accumulate in a lesion in a large amount (agent fluorescence) have been proposed. In the diagnostic technique utilizing agent fluorescence, fluorescent agents having a property of accumulating in tumor tissue, for example hematoporphyrin derivative, Photofrin derivative, indocyanine green derivative labeled antibody, or the like, are used.

When tumor tissue is to be identified by this diagnostic technique, first of all, fluorescent agent, such as those described above, is injected into the living organism before conducting the diagnosis. Then, after the fluorescent agent has accumulated in the tumor tissue, the endoscope is inserted to irradiate the interior of the body cavity with excitation light having an excitation wavelength band of the fluorescent agent, thus causing fluorescence to be produced by the fluorescent agent accumulated in the tumor tissue. The fluorescence emitted from the fluorescent agent accumulated in the tumor tissue is received by the endoscope and acquired as a fluorescence image. Accordingly, a person conducting the diagnosis diagnoses a high-luminance region in the fluorescence image as the tumor tissue. Several techniques have been proposed with regard to an endoscope apparatus that can be applied to such a diagnostic technique (for example, see Patent Documents 1 and 2).

The endoscope apparatus disclosed in Patent Document 1 is a fluorescence endoscope apparatus in which hematoporphyrin derivative is used as the fluorescent agent. Furthermore, the endoscope apparatus disclosed in Patent Document 2 is a fluorescence endoscope apparatus in which indocyanine green derivative labeled antibody is used as the fluorescent agent. These fluorescence endoscope apparatuses disclosed in Patent Document 1 and Patent Document 2 can acquire only the fluorescence from the body cavity by providing a fluorescence filter or the like for reflecting excitation light onto the whole area of an image-acquisition unit that acquires the fluorescence.

Patent Document 1:

Japanese Unexamined Patent Application Publication No. Patent Document 2:

Japanese Unexamined Patent Application Publication No.

### DISCLOSURE OF INVENTION

With the endoscope apparatuses of Patent Document 1 and Patent Document 2, excitation light emitted from a light source provided at the base end of an insertion portion that is inserted into the body cavity is guided to the distal end of the insertion portion through a light guide fiber provided within the insertion portion to irradiate the inner wall of the body cavity (the body cavity inner wall). While the excitation light is passing through the light guide fiber, noise light, such as Raman scattered light or autofluorescence, is produced within the light guide fiber (hereinafter, referred to as noise light originating in the light guide portion), and the fluorescence to be acquired by the image-acquisition unit becomes contaminated with the noise light.

That is to say, since the noise light originating from light guiding, produced due to excitation by the excitation light, includes light having longer wavelengths than the wavelength of the excitation light, it cannot be removed with the fluorescence filter at the preceding stage of the image-acquisition unit and will end up reaching the image-acquisition unit. Thus, since the noise light originating in the light guide portion, which is reflected from normal tissue, is acquired by the image-acquisition unit together with the fluorescence produced from the fluorescent agent in the body cavity, it becomes difficult to distinguish between lesion tissue, such as tumor tissue, and normal tissue in the acquired fluorescence image.

The present invention provides a fluorescence endoscope apparatus that can remove the influence of noise light originating in the light guide portion, produced at a light guide portion, by simple calculations, and that can acquire a clear fluorescence image that makes distinction between lesion tissue and normal tissue easier.

### BRIEF SUMMARY OF THE INVENTION

A first aspect of the present invention is a fluorescence endoscope apparatus including an insertion portion for insertion into a body cavity; a light source unit that is disposed at a base end of the insertion portion and that emits excitation light and reference light that contains at least a part of the wavelength band of fluorescence produced by the excitation light; a light guide portion that guides the excitation light and the reference light emitted from the light source unit to a distal end of the insertion portion; an irradiation control unit that switches between a first irradiation state in which the excitation light guided by the light guide portion is radiated onto an inner wall of the body cavity and a second irradiation state in which the reference light is radiated onto the inner wall of the body cavity; an image-acquisition unit that acquires reflected light of the reference light and the fluorescence returning from the inner wall of the body cavity to the insertion portion; and an image computing unit that generates a fluorescence image signal by calculating the difference between a first image-acquisition signal acquired by the image-acquisition unit in the first irradiation state and a second image-acquisition signal acquired in the second irradiation state.

According to the above described first aspect, by the operation of the irradiation control unit, the excitation light is radiated onto the inner wall of the body cavity in the first irradiation state, and the reference light is radiated in the second irradiation state. The excitation light is guided from the light source unit, which is disposed at the base end of the insertion portion, to the distal end through the light guide portion and radiated onto the inner wall of the body cavity, thereby exciting the fluorescent agent in the inner wall of the body cavity and producing fluorescence therefrom. The fluorescence produced is acquired by the image-acquisition unit and obtained as the first image-acquisition signal.

In this case, the noise light originating in the light guide portion, which is generated while the excitation light is passing through the light guide portion, is also radiated onto the inner wall of the body cavity, reflected at the surface of the inner wall of the body cavity, and returned as reflected light. The noise light originating in the light guide portion contains a wavelength band equivalent to that of fluorescence at longer wavelengths than the excitation light, and it is acquired by the image-acquisition unit even if the fluorescence filter is provided. Therefore, the first image-acquisition signal contains the fluorescence from the fluorescent agent in the inner wall of the body cavity and the signal due to the noise light originating in the light guide portion.

On the other hand, the reference light is also guided from the light source unit, which is disposed at the base end of the insertion portion, to the distal end through the light guide portion, and when radiated onto the inner wall of the body cavity, it is reflected at the surface thereof and returned as the reflected light. Since the reference light contains at least a part of the wavelength band of the fluorescence produced with the excitation light, it is acquired by the image-acquisition unit and obtained as the second image-acquisition signal even if the fluorescence filter is provided.

Therefore, with the operation of the image computing unit, it is possible to produce a clear fluorescence image, from which the noise light originating in the light guide portion has been removed, by calculating the difference between the first image-acquisition signal and the second image-acquisition signal to obtain the fluorescence image signal, thus compensating for an intensity component of the noise light originating in the light guide portion contained in the first image-acquisition signal and an intensity component of the reflected light of the reference light that is the second image-acquisition signal, if they are equivalent.

In the above described first aspect, the fluorescence endoscope apparatus may further include a noise light detection unit that detects the light level of noise light originating in the light guide portion, which is produced by guiding the excitation light in the light guide portion; and a reference-light adjusting unit that adjusts the light level of the reference light such that the light level of the reference light becomes equal to the light level of the noise light originating in the light guide portion, detected by the noise light detection unit.

By doing so, by the operation of the reference-light adjusting unit, the light level of the reference light is adjusted so that it becomes equal to the light level of the noise light originating in the light guide portion, which is detected by the noise light detection unit. Therefore, in the image computing unit, it is possible to generate a fluorescence image from which the intensity component of the noise light originating in the light guide portion has been removed, merely by subtracting the second image-acquisition signal from the first image-acquisition signal.

Further, in the above described structure, the reference-light adjusting unit may include a filter that varies the transmitted light level of the reference light.

By doing so, it is possible to easily match the light level of the reference light and the light level of the noise light originating in the light guide portion.

A second aspect of the present invention is a fluorescence endoscope apparatus including an insertion portion for insertion into a body cavity; a light source unit that is disposed at a base end of the insertion portion and that emits excitation light and reference light that contains at least a part of the wavelength band of fluorescence produced by the excitation light; a light guide portion that guides the excitation light and the reference light emitted from the light source unit to a distal end of the insertion portion; an irradiation control unit that switches between a first irradiation state in which the excitation light guided by the light guide portion is radiated onto an inner wall of the body cavity and a second irradiation state in which the reference light is radiated onto the inner wall of the body cavity; an image-acquisition unit that acquires reflected light of the reference light and the fluorescence returning from the inner wall of the body cavity to the insertion portion; and an image computing unit that generates a fluorescence image signal by calculating the difference between a corrected image-acquisition signal obtained by multiplying a first image-acquisition signal acquired by the image-acquisition unit in the first irradiation state by a correction factor set based on the intensity of the reference light and a second image-acquisition signal acquired by the image-acquisition unit in the second irradiation state.

According to the above described second aspect, by the operation of the irradiation control unit, the excitation light is radiated onto the inner wall of the body cavity in the first irradiation state, and the reference light is radiated in the second irradiation state. The excitation light is guided from the light source unit, which is disposed at the base end of the insertion portion, to the distal end through the light guide portion and radiated onto the inner wall of the body cavity, thereby exciting the fluorescent agent in the inner wall of the body cavity and producing fluorescence therefrom. The fluorescence produced is acquired by the image-acquisition unit and acquired as the first image-acquisition signal.

In this case, the noise light originating in the light guide portion, which is generated while the excitation light is passing through the light guide portion, is also radiated onto the inner wall of the body cavity, reflected at the surface of the inner wall of the body cavity, and returned as reflected light. The noise light originating in the light guide portion contains a wavelength band equivalent to that of the fluorescence at longer wavelengths than the excitation light, and it is acquired by the image-acquisition unit even if the fluorescence filter is provided. Therefore, the first image-acquisition signal contains the fluorescence from the fluorescent agent in the inner wall of the body cavity and the signal due to the noise light originating in the light guide portion.

On the other hand, the reference light is also guided from the light source unit, which is disposed at the base end of the insertion portion, to the distal end through the light guide portion, and when radiated onto the inner wall of the body cavity, it is reflected at the surface thereof and returned as the reflected light. Since the reference light contains at least a part of the wavelength band of the fluorescence produced with the excitation light, it is acquired by the image-acquisition unit and acquired as the second image-acquisition signal even if the fluorescence filter is provided.

Although the intensity component of the noise light originating in the light guide portion contained in the first image-acquisition signal and the intensity component of the reflected light of the reference light contained in the second image-acquisition signal tend to be different in many cases, it is possible to match the intensity component of the noise light originating in the light guide portion contained in the corrected image-acquisition signal with the second image-acquisition signal by multiplying the first image-acquisition signal by the correction factor, which is set in accordance with the intensity of the reference light, to derive the corrected image-acquisition signal.

Therefore, with the operation of the image computing unit, it is possible to produce a clear fluorescence image from which the intensity component of the noise light originating in the light guide portion has been removed, by deriving the corrected image-acquisition signal, and calculating the difference between the corrected image-acquisition signal and the second image-acquisition signal to acquire the fluorescence image signal.

In the above described second aspect, the fluorescence endoscope apparatus may further include a noise light detection unit that detects the light level of noise light originating in the light guide portion, which is produced by guiding the excitation light in the light guide portion; and a correction-factor setting unit that sets the correction factor based on the ratio of the light level of the noise light originating in the light guide portion, detected by the noise light detection unit, to the light level of the reference light.

By doing so, with the operation of the correction-factor setting unit, it is possible to obtain the correction factor for matching the intensity component of the noise light originating in the light guide portion contained in the corrected image-acquisition signal with the second image-acquisition signal with superior precision. Therefore, it is possible to generate a clear fluorescence image from which the intensity component of the noise light originating in the light guide portion has been sufficiently removed.

The present invention affords an advantage in that it is possible to remove the effect of the noise light originating in the light guide portion, generated in the light guide portion, by simple calculations, and to acquire a clear fluorescence image that facilitates distinction between lesion tissue and normal tissue.

### BRIEF DESCRIPTION OF DRAWINGS

FIG. 1 is a diagram showing the overall configuration of a fluorescence endoscope apparatus according to a first embodiment of the present invention.

FIG. 2 is a time chart of light emitted from a light source unit of the fluorescence endoscope apparatus in FIG. 1.

FIG. 3 is a flow chart explaining processing in an image computing unit of the fluorescence endoscope apparatus in FIG. 1.

FIG. 4 is a diagram showing a modification of the light source unit of the fluorescence endoscope apparatus in FIG. 1.

FIG. 5 is a diagram showing a filter turret used in the light source unit in FIG. 4.

FIG. 6 is a time chart of light emitted from the light source unit in a modification of FIG. 4.

FIG. 7 is a diagram showing a transmittance characteristic of the filter turret in FIG. 5.

FIG. 8 is a flow chart explaining processing in the image computing unit for the fluorescence endoscope apparatus with the light source unit in FIG. 4.

FIG. 9 is a diagram showing another modification of the light source unit of the fluorescence endoscope apparatus in FIG. 1.

FIG. 10 is a time chart of light emitted from the light source unit in the modification of FIG. 9.

FIG. 11 is a flow chart explaining processing in the image computing unit for the fluorescence endoscope apparatus with the light source unit in FIG. 9.

FIG. 12 is a diagram showing the overall configuration of another modification of the fluorescence endoscope apparatus in FIG. 1.

FIG. 13 is a diagram showing the overall configuration of a fluorescence endoscope apparatus according to a second embodiment of the present invention.

FIG. 14 is a flow chart explaining processing in an image computing unit of the fluorescence endoscope apparatus in FIG. 13.

### EXPLANATION OF REFERENCE SIGNS

A: body cavity inner wall (inner wall of body cavity)
1, 1′: fluorescence endoscope apparatus
2: insertion portion
3: light source unit
6: light guide fiber (light guide portion)
9: image-acquisition unit
14: irradiation control unit
29: image computing unit
42: light level detector (noise light detection unit)
43: correction-factor calculating unit (correction-factor setting unit)

### BEST MODE FOR CARRYING OUT THE INVENTION

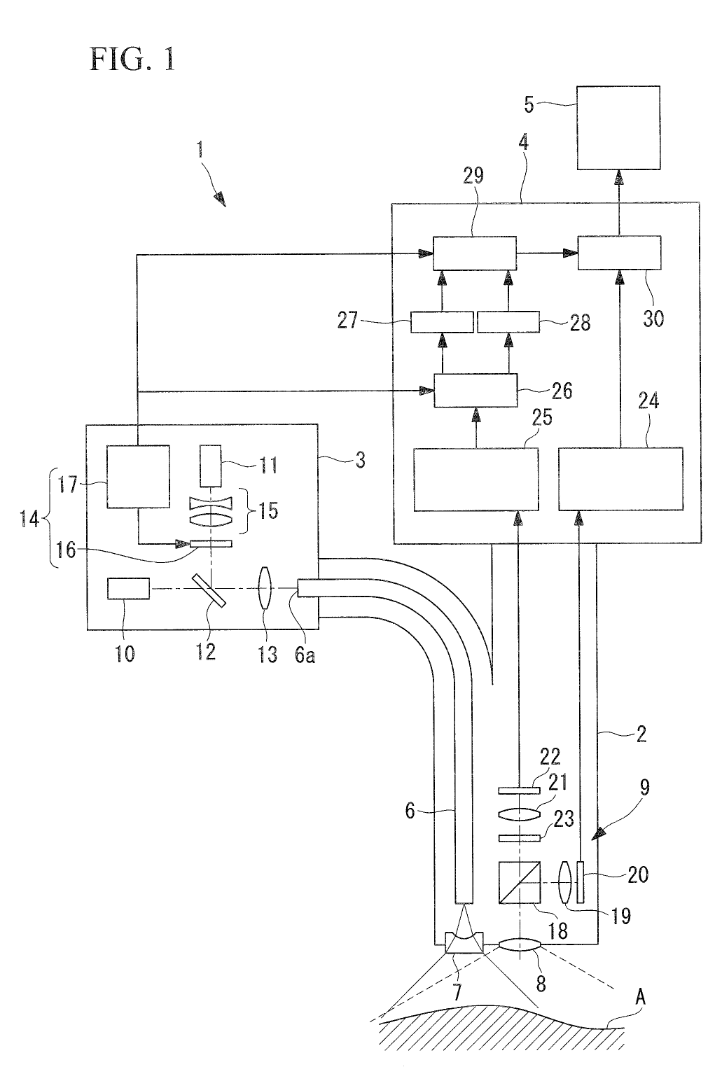

A fluorescence endoscope apparatus 1 according to a first embodiment of the present invention will be described below with reference to FIGS. 1 to 3.

As shown in FIG. 1, the fluorescence endoscope apparatus 1 according to the present embodiment includes a long thin insertion portion 2 that is inserted into a body cavity, a light source unit 3 and an image processing unit 4 arranged at the base end of the insertion portion 2, and a monitor 5 connected to the image processing unit 4.

The insertion portion 2 is provided with a light guide fiber 6 that is disposed along the longitudinal direction of the insertion portion 2 from the base end to the distal end thereof and that guides light from the light source unit 3, an illumination optical system 7 that is disposed at the distal end of the light guide fiber 6 and that spreads the light that has been guided and radiates it onto the body cavity inner wall A, an objective lens 8 that collects light returning from the body cavity inner wall A, and an image-acquisition unit 9 that acquires the light collected by the objective lens 8.

The light source unit 3 is provided with a white light source 10 that emits white light and excitation light, a reference light source 11 that emits reference light, a dichroic mirror 12 that combines the white light, the excitation light, and the reference light onto the same optical path, a coupling lens 13 that focuses the combined white light, the excitation light, and/or the reference light onto an entrance end 6a of the light guide fiber 6, and an irradiation control unit 14 that switches between two irradiation states. In the figure, reference sign 15 is a beam expander that adjusts the beam diameter of the reference light.

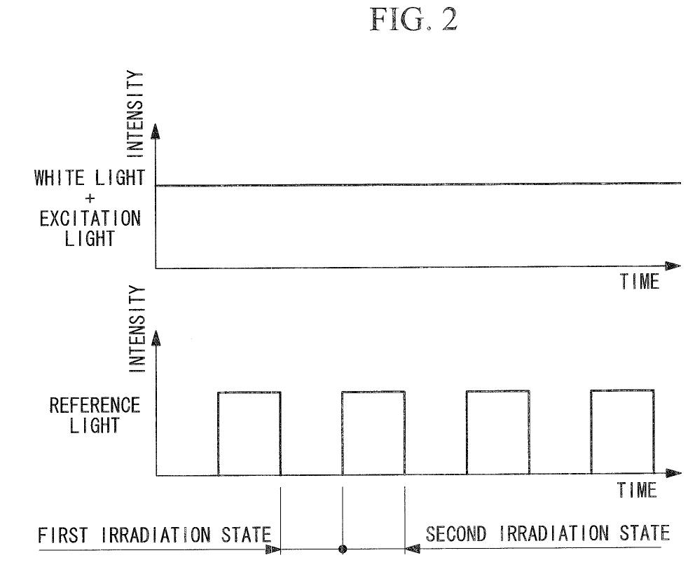

The irradiation control unit 14 includes a light chopper 16 that is disposed between the beam expander 15 and the dichroic mirror 12 and that opens and closes the optical path by turning on and off, and a chopper drive unit 17 that controls the light chopper 16 on and off. As shown in FIG. 2, by turning on and off the reference light to be combined by the operation of the chopper drive unit 17, it is possible to switch between a first irradiation state in which the white light and the excitation light are radiated and a second irradiation state in which the white light, the excitation light, and the reference light are radiated.

The image-acquisition unit 9 includes a dichroic mirror 18 that divides the light collected by the objective lens 8 into the white light and the fluorescence, a focusing lens 19 that focuses the white light divided by the dichroic mirror 18, a white-light image-acquisition device 20, such as a CCD, that acquires the white light focused by the focusing lens, a focusing lens 21 that focuses the fluorescence divided by the dichroic mirror 18, and a fluorescence image-acquisition device 22, such as CCD, that acquires the fluorescence focused by the focusing lens 21. In the figure, reference sign 23 is an excitation light cut filter that blocks the excitation light contained in the fluorescence.

The image processing unit 4 includes a white-light image generating unit 24 that generates a white-light image signal based on an image-acquisition signal of the white light acquired by the white-light image-acquisition device 20, a fluorescence image generating unit 25 that generates a fluorescence image signal based on an image-acquisition signal of the fluorescence acquired by the fluorescence image-acquisition device 22, a fluorescence-image signal separating unit 26 that separates a first image signal acquired by the fluorescence image-acquisition device 22 in the first irradiation state and a second image signal acquired by the fluorescence image-acquisition device 22 in the second irradiation state, first and second memories 27 and 28 that store the separated first and second image signals, respectively, an image computing unit 29 that conducts computational processing using the first and second image signals stored in the first and second memories 27 and 28, and an image combining unit 30 that combines the fluorescence image signal generated as a result of the calculation conducted in the image computing unit 29 and the white-light image signal generated in the white-light image generating unit 24 and outputs them to a monitor 5.

The fluorescence-image signal separating unit 26 is configured so as to receive a signal showing the driven state of the light chopper 16, which signal is output from the chopper drive unit 17, and to switch the output to the first and second memories 27 and 28 in synchronization with this signal.

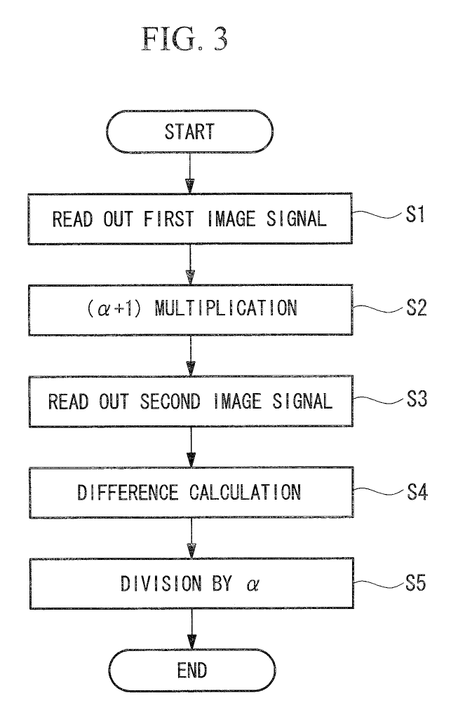

The image computing unit 29 is provided with a predetermined factor α, and, as shown in FIG. 3, is configured so as to, first of all, read out the first image signal acquired in the first irradiation state and stored in the first memory 27 (Step S1), and to multiply the read out first image signal by the correction factor (α+1) thus calculating the corrected image signal (Step S2).

Next, the second image signal acquired in the second irradiation state and stored in the second memory 28 is read out (Step S3), and the read out second image signal is subtracted from the corrected image signal (Step S4). Then, the signal obtained via subtraction is divided by the factor α (Step S5).

The timings of reading out the first and second memories 27 and 28 are set in synchronization with the signal showing the driven state of the light chopper 16 that is output from the chopper drive unit 17.

That is to say, since the first image signal contains a fluorescence signal Sf produced from the body cavity inner wall A and a signal Sn of the noise light originating in the light guide portion, which is reflected from the body cavity inner wall A, it is expressed as (Sf+Sn). In addition, since the second image signal further contains a reference light signal Sr reflected from the body cavity inner wall A, it is expressed as (Sf+Sn+Sr).

Accordingly, expressing the above described procedure in the image computing unit 29 as formulae, the fluorescence image signal F finally acquired is:

F=((α+1)(Sf+Sn)−(Sf+Sn+Sr))/α (1).

Modifying equation (1) yields:

F=(α(Sf+Sn)−Sr)/α (2).

By obtaining it experimentally, as the factor α, the ratio of the signal Sn of the noise light originating in the light guide portion to the reference light signal Sr, a=Sr/Sn (3), equation (2) can be modified to:

F=(α(Sf+Sn)−αSn)/α=Sf (4).

That is to say, as shown in equation (4), it is possible to easily acquire the fluorescence signal Sf that does not contain the signal Sn of the noise light originating in the light guide portion as the fluorescence image signal F by calculation.

With the thus-configured fluorescence endoscope apparatus 1 according to this embodiment, it is possible to switch between and alternately radiate the excitation light and the reference light with a wavelength band that contains the same wavelength as that of the fluorescence, and to simply and rapidly generate the fluorescence image signal F that does not contain the signal Sn of the noise light originating in the light guide portion based on the obtained two types of image signals. Therefore, it is possible to display a fluorescence image in which the fluorescence produced from the affected area of the body cavity inner wall A is brightened and made distinguishable on the monitor 5, thus distinguishing between the affected area and the normal area and allowing accurate diagnosis.

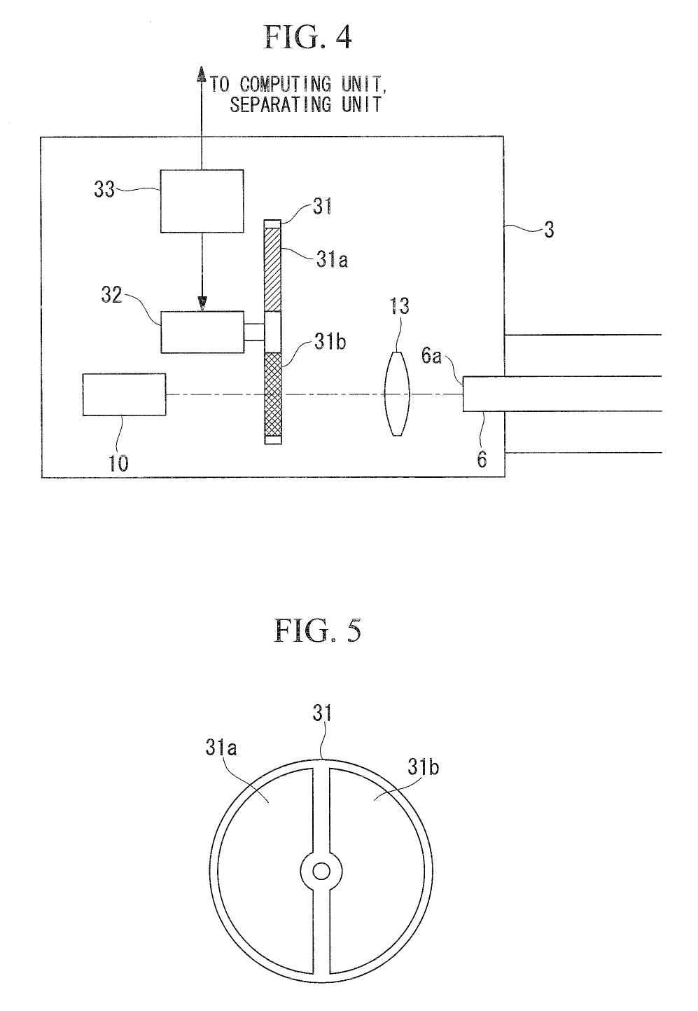

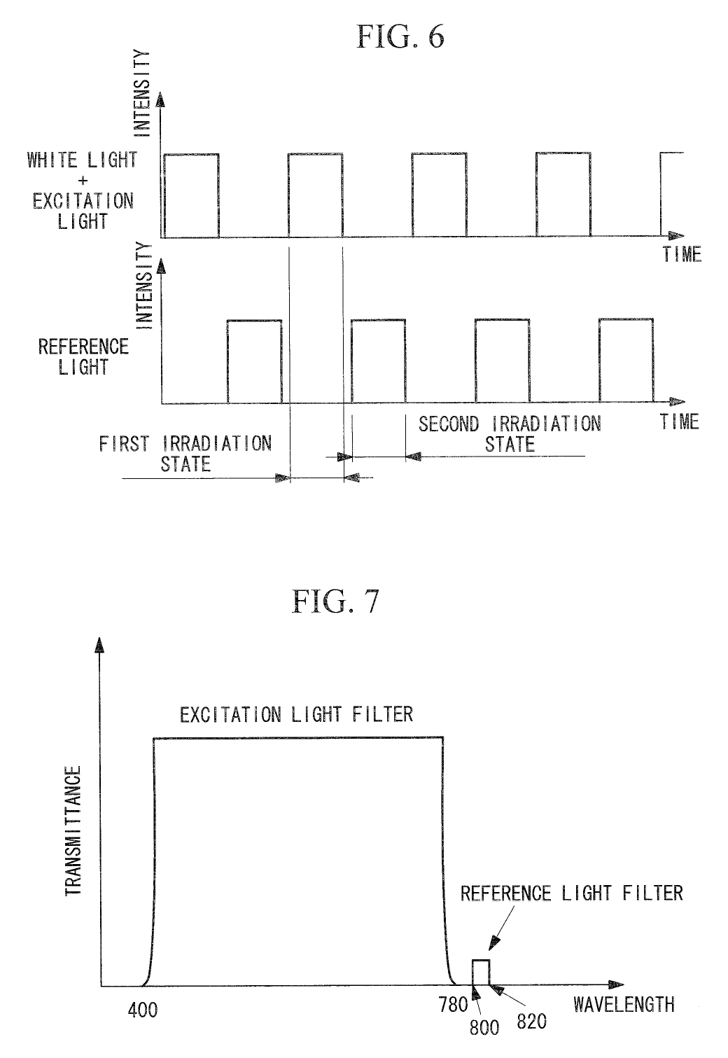

Although, in the present embodiment, the white light and excitation light from the white light source 10 are irradiated continuously, and the reference light from the reference light source 11 is irradiated intermittently by driving the light chopper 16, thereby switching between the first irradiation state and the second irradiation state, instead of this, as shown in FIGS. 4 and 5, a filter turret 31 provided with an excitation light filter 31a that transmits the excitation light and a reference light filter 31b that transmits the reference light may be employed. That is to say, by rotating the filter turret 31 in the optical path of the white light source, and arranging the excitation light filter 31a and the reference light filter 31b in the optical path in an alternating switching manner, as shown in FIG. 6, the excitation light and the reference light can be alternately switched and selectively transmitted from the white light emitted from the white light source 10.

In this case, a signal output from a motor drive unit 33 of a motor 32 that rotationally drives the filter turret 31 may be used as the signal for synchronously driving the fluorescence-image signal separating unit 26 and the image computing unit 29.

In addition, since the light level of the fluorescence produced with respect to the light level of the excitation light is very minute, and the light level of the noise light originating in the light guide portion is also minute, the light level of the reference light to be radiated to remove the noise light originating in the light guide portion is also required to have a light level equal to the noise light originating in the light guide portion. Thus, as shown in FIG. 7, it is preferred to set the transmittance of the reference light filter 31b sufficiently small relative to the transmittance of the excitation light filter 31a. The numerical values showing the wavelength in FIG. 7 are only examples.

At this time, since the first image signal acquired in the first irradiation state contains the fluorescence signal Sf produced from the body cavity inner wall A and the signal Sn of the noise light originating in the light guide portion, which is reflected from the body cavity inner wall A, it is expressed as (Sf+Sn). In addition, since the second image signal is the reference light signal Sr alone, it is expressed as Sr.

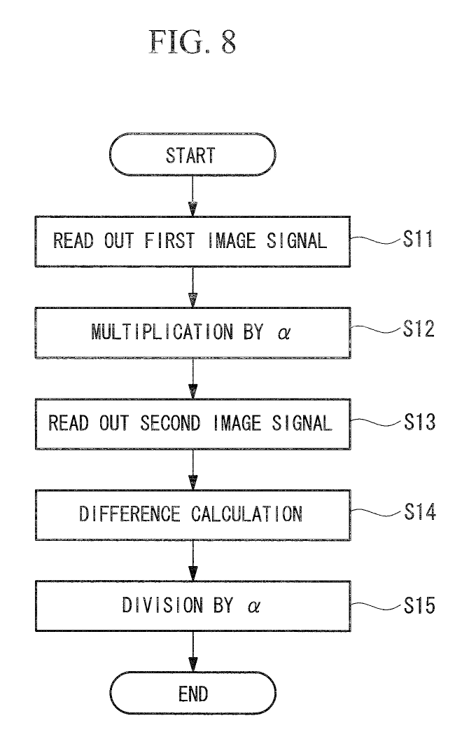

Therefore, as shown in FIG. 8, as the fluorescence image signal F, it is possible to obtain:

$$\begin{matrix}F = ( α ( Sf + Sn ) - Sr ) / α \\ = ( α Sf + α Sn - α Sn ) / α \\ = Sf\end{matrix}$$

by reading out the first image signal (Step S11), generating the corrected image signal by multiplying the read out first image signal by the correction factor α (Step S12), reading out the second image signal (Step S13), subtracting the second image signal from the corrected image signal (Step S14), and further, dividing the entirety by the correction factor α (Step S15). Therefore, also by doing so, the fluorescence signal Sf that does not contain the signal Sn of the noise light originating in the light guide portion can be easily acquired by calculation, as the fluorescence image signal F.

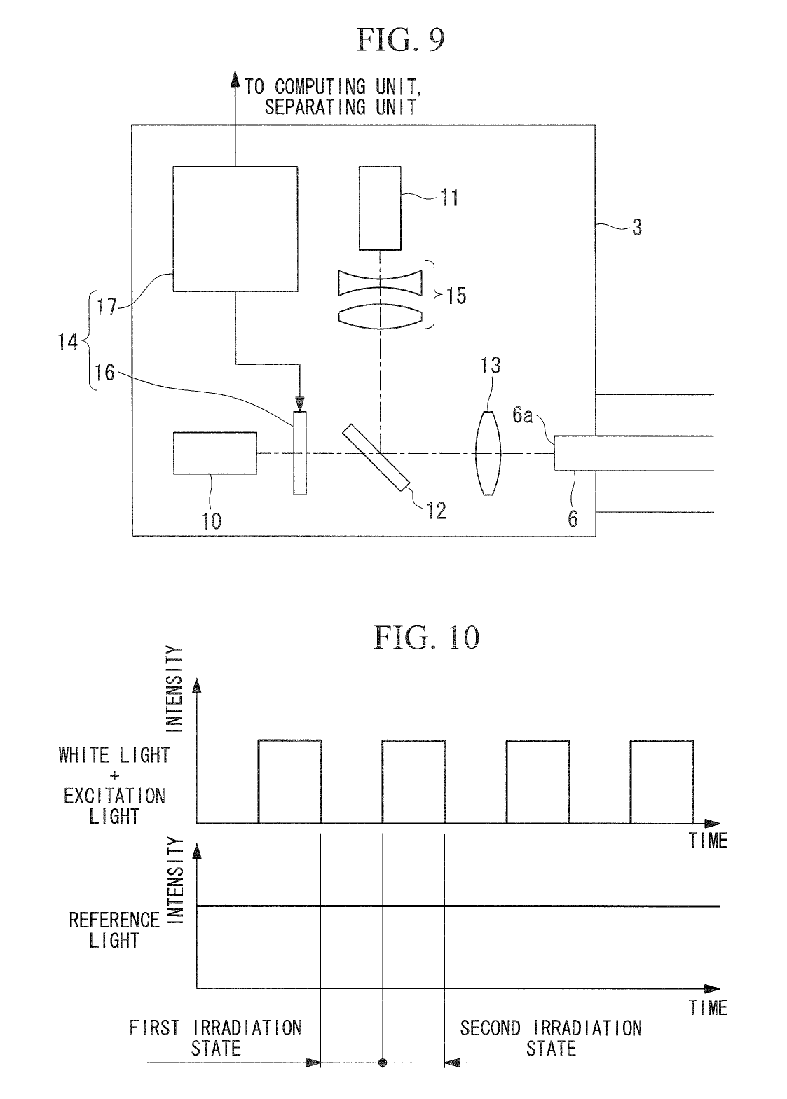

In addition, as shown in FIG. 9, instead of turning the reference light on and off with the light chopper 16, the white light and the excitation light may be turned on and off with the light chopper 16.

In this case, as shown in FIG. 10, since the reference light is radiated continuously and the white light and the excitation light are irradiated intermittently, the first image signal acquired in the first irradiation state is the reference light Sr alone, expressed as Sr. Furthermore, since the second image signal acquired in the second irradiation state contains, in addition to the reference light Sr, the fluorescence signal Sf produced from the body cavity inner wall A and the signal Sn of the noise light originating in the light guide portion reflected from the body cavity inner wall A, it is expressed as (Sf+Sn+Sr).

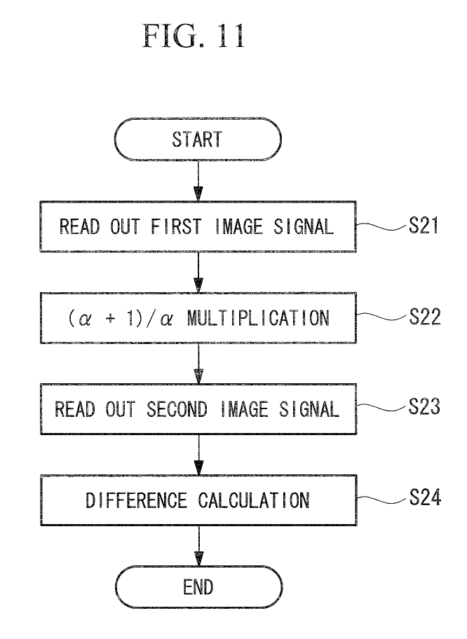

Therefore, as shown in FIG. 11, as the fluorescence image signal F, it is possible to obtain:

$$\begin{matrix}F = ( Sf + Sn + Sr ) - ( ( α + 1 ) / α ) Sr \\ = ( Sf + ( α + 1 ) Sn ) - ( α + 1 ) Sn \\ = Sf\end{matrix}$$

by reading out the first image signal (Step S21), generating the corrected image signal by multiplying the read out first image signal by the correction factor (α+1)/α (Step S22), reading out the second image signal (Step S23), and subtracting the corrected image signal from the second image signal (Step S24). Therefore, also by doing so, the fluorescence signal Sf that does not contain the signal Sn of the noise light originating in the light guide portion can be easily acquired by calculation, as the fluorescence image signal F.

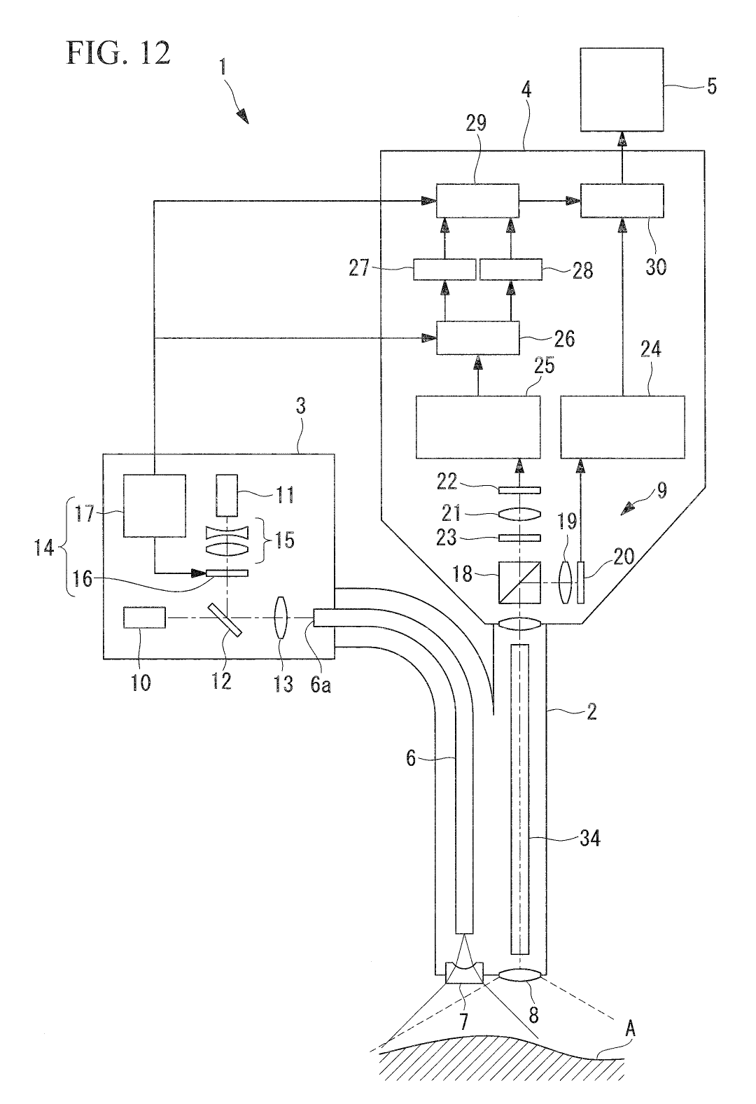

In addition, in the present embodiment, although the image-acquisition unit 9 is disposed at the distal end part of the insertion portion 2, instead of this, as shown in FIG. 12, an image guide fiber 34 that transmits the light collected by the objective lens 8 may be disposed in the insertion portion 2, and the image-acquisition unit 9 may be disposed within the image processing unit 4 at the base end of the insertion portion 2. By doing so, the insertion portion 2 can be made narrower.

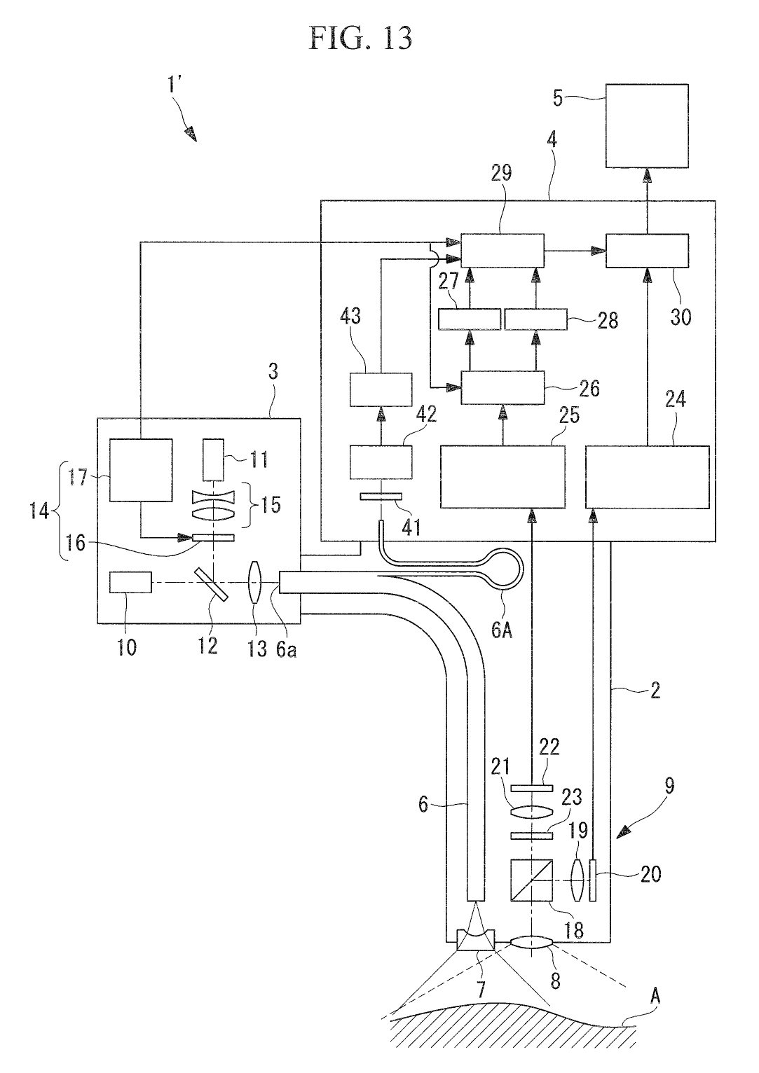

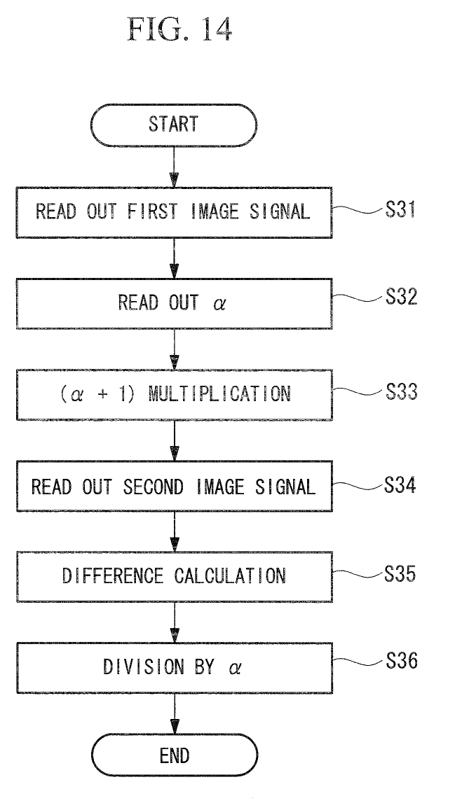

Next, a fluorescence endoscope apparatus 1′ according to a second embodiment of the present invention will be described below with reference to FIGS. 13 and 14.

In the description of the present embodiment, elements having the same configuration as those in the fluorescence endoscope apparatus 1 according to the first embodiment described above are given the same reference signs, and a description thereof is omitted.

As shown in FIG. 13, in the fluorescence endoscope apparatus 1′ according to the present embodiment, the distal end of a light guide fiber portion 6A branched from a part of the light guide fiber 6 is connected to the image processing unit 4, and the image processing unit 4 is provided with an excitation light cut filter 41, a light level detector 42, and a correction-factor calculating unit 43.

The length of the branched light guide fiber portion 6A is preferably the same as the length of the other part of the light guide fiber 6 that extends to the distal end of the insertion portion 2.

The excitation light cut filter 41 is configured so as to be able to block the excitation light being transmitted through the branched light guide fiber portion 6A and to transmit only the noise light originating in the light guide portion generated in the light guide fiber portion 6A. The light level detector 42 is, for example, a photodiode.

The correction-factor calculating unit 43 is configured so as to store the intensity of the predetermined reference light signal Sr and to calculate the factor α using equation (3) by performing division using the intensity of the signal Sn of the noise light originating in the light guide portion, which is detected by the light level detector 42.

With the thus-configured fluorescence endoscope apparatus 1′ according to the present embodiment, the image computing unit 29 reads out the first image signal and the factor α from the first memory 27 and the correction-factor calculating unit 43 in synchronization with a chopper drive signal received from the chopper drive unit 17 (Steps S31, S32), and calculates the corrected image signal by multiplying the read out first image signal by the correction factor (α+1) (Step S33).

Next, the second image signal stored in the second memory 28 is read out (Step S34), and the read out second image signal is subtracted from the corrected image signal (Step S35). Then, the signal obtained via subtraction is divided by the factor α (Step S36). By doing so, the fluorescence signal Sf that does not contain the signal Sn of the noise light originating in the light guide portion is produced, according to equations (1) to (4).

According to this embodiment, since the factor α is successively calculated by detecting the signal Sn of the noise light originating in the light guide portion, there is an advantage in that it is possible to more reliably remove the noise light originating in the light guide portion from the fluorescence image signal by calculating the factor α with superior precision, even when the signal Sn of the noise light originating in the light guide portion is varied due to changes in the conditions in the light guide fiber 6, for example, temperature changes and the like.

A variable filter (not shown in the figure), such as an acousto-optic device, that adjusts the reference light such that the intensity of the reference light becomes equal to the intensity of the noise light originating in the light guide portion, generated by the light guide fiber portion 6A, may be provided. By doing so, the factor α can be constantly maintained at 1, which provides an advantage in that it is possible to make the calculation easier by simplifying the correction factor to be used in the calculation.

1. A fluorescence endoscope apparatus comprising:
an insertion portion for insertion into a body cavity,
a light source unit that is disposed at a base end of the insertion portion and that emits excitation light and reference light that contains at least a part of the wavelength band of fluorescence produced by the excitation light;
a light guide portion that guides the excitation light and the reference light emitted from the light source unit to a distal end of the insertion portion;
an irradiation control unit that switches between a first irradiation state in which the excitation light guided by the light guide portion is radiated onto an inner wall of the body cavity and a second irradiation state in which the reference light is radiated onto the inner wall of the body cavity;
an image-acquisition unit that acquires reflected light of the reference light and the fluorescence returning from the inner wall of the body cavity to the insertion portion; and
an image computing unit that generates a fluorescence image signal by calculating the difference between a first image-acquisition signal acquired by the image-acquisition unit in the first irradiation state and a second image-acquisition signal acquired in the second irradiation state.

2. A fluorescence endoscope apparatus according to claim 1, further comprising:
a noise light detection unit that detects the light level of noise light originating in the light guide portion, which is produced by guiding the excitation light in the light guide portion; and
a reference-light adjusting unit that adjusts the light level of the reference light such that the light level of the reference light becomes equal to the light level of the noise light originating in the light guide portion, detected by the noise light detection unit.

3. A fluorescence endoscope apparatus according to claim 2, wherein the reference-light adjusting unit comprises a filter that varies the transmitted light level of the reference light.

4. A fluorescence endoscope apparatus comprising:
an insertion portion for insertion into a body cavity;
a light source unit that is disposed at a base end of the insertion portion and that emits excitation light and reference light that contains at least a part of the wavelength band of fluorescence produced by the excitation light;
a light guide portion that guides the excitation light and the reference light emitted from the light source unit to a distal end of the insertion portion;
an irradiation control unit that switches between a first irradiation state in which the excitation light guided by the light guide portion is radiated onto an inner wall of the body cavity and a second irradiation state in which the reference light is radiated onto the inner wall of the body cavity;
an image-acquisition unit that acquires reflected light of the reference light and the fluorescence returning from the inner wall of the body cavity to the insertion portion; and
an image computing unit that generates a fluorescence image signal by calculating the difference between a corrected image-acquisition signal obtained by multiplying a first image-acquisition signal acquired by the image-acquisition unit in the first irradiation state by a correction factor set based on the intensity of the reference light and a second image-acquisition signal acquired by the image-acquisition unit in the second irradiation state.

5. A fluorescence endoscope apparatus according to claim 4 further comprising:
a noise light detection unit that detects the light level of noise light originating in the light guide portion, which is produced by guiding the excitation light in the light guide portion; and
a correction-factor setting unit that sets the correction factor based on the ratio of the light level of the noise light originating in the light guide portion, detected by the noise light detection unit, to the light level of the reference light.
